(19) United States
(12) Patent Application Publication
(10) Pub. No.: US 2025/0194522 A1
(43) Pub. Date: Jun. 19, 2025

(54) PEANUT CULTIVAR 'IPG 913'

(71) Applicant: International Peanut Group LLC, Brownfield, TX (US)

(72) Inventors: Dylan Quincy Wann, Brownfield, TX (US); Jimmy D. Gregory, Brownfield, TX (US)

(21) Appl. No.: 18/982,050

(22) Filed: Dec. 16, 2024

Related U.S. Application Data
(60) Provisional application No. 63/610,788, filed on Dec. 15, 2023.

(51) Int. Cl. A01H 6/54; A01H 5/10

(52) U.S. Cl. CPC A01H 6/541; A01H 5/10

(57) ABSTRACT

A peanut cultivar designated IPG 913 is disclosed herein. The present invention provides seeds, plants, and plant parts derived from peanut cultivar IPG 913. Further, it provides methods for producing a peanut plant by crossing IPG 913 with itself or another peanut variety. The invention also encompasses any peanut seeds, plants, and plant parts produced by the methods disclosed herein, including those in which additional traits have been transferred into IPG 913 through genetic engineering, gene editing, mutagenesis, or by breeding IPG 913 with another peanut cultivar.

### CROSS-REFERENCE TO RELATED APPLICATIONS

This application claims priority to U.S. Provisional Application No. 63/610,788 filed on Dec. 15, 2023, the contents of which are incorporated by reference in its entirety.

### BACKGROUND OF THE INVENTION

The present invention relates to a new and distinctive peanut (Arachis hypogaea L.) cultivar, designated ‘IPG 913.’

The peanut is an annual herbaceous plant of the legume family. Originally cultivated in South America and the eastern slopes of the Andes mountains, peanut is now grown worldwide in the tropic and temperate zones and is recognized as one of the major oilseed crops and as a rich source of protein.

The peanut plant grows best in light, sandy soil and requires four to five months of warm weather and an annual rainfall of 20 to 39 inches, or the equivalent in irrigation water. The pea-like yellow flowers form in axillary clusters and only bloom for a short time. Following self-pollination, the stalk at the base of the ovary, called the pedicel, elongates rapidly and turns downward to bury the fruits one to several inches below the ground surface. The peanut pods complete their development 120 to 150 days after planting. During harvest, the entire plant including the roots is removed from the soil.

Peanut is an important and valuable crop. Peanut is particularly susceptible to viruses and fungi during growth and storage. Indeed, diseases are major constraints to peanut production worldwide.

In the United States, spotted wilt can be considered an invasive disease of peanut caused by the pathogen Tomato Spotted Wilt Tospovirus (TSWV) (Demski, J. W. and D. V. R. Reddy, Tomato spotted wilt and peanut bud necrosis. Compendium of Peanut Diseases, 2^{nd} Edition, pp. 53-54 (1997)). The disease is transferred to plants by thrips carrying the virus, the two most common species being Tobacco thrips (Frankliniella fusca) and Western flower thrips (Frankliniella occidentalis). These tiny insects are also referred to as thunderflies, thunderbugs, storm flies, thunderblights, and corn lice. While there are still unknown factors influencing the incidence of TSWV, research has shown that planting date, plant population, row patterns, and tillage can affect the severity of disease.

The initial symptoms of spotted wilt can appear as early as 21 days after the seedlings emerge. Early symptoms include brown speckles on the underside of leaves, along with yellow ring-spotting and mottling on the upper side of the leaf. Leaves may also be wilted or flaccid, and new leaves are about half their normal size, crinkled, and display a range of symptoms including chlorosis, concentric chlorotic ring-spots, ring-spots with green centers, chlorotic line patterns, and general mottling. A downward twisting of leaf petioles and some terminals can also be seen at this stage. Brown, necrotic spots or streaks may also be present on the leaf petiole and stem. Stunting is commonly observed on plants infected at the seedling stage, and few pods are set. Seeds that are produced by TSWV infected plants are smaller than normal and have mottled red to brown seed coats that are often cracked and show poor germination. Late season TSWV infections are characterized by a decline in plant vigor, yellowing of the foliage, and collapse of the vines. Faint ring-spot or line patterns may be seen on the youngest leaves, and the root systems of these plants are often discolored and partially rotted. Severe infections may result in plant death.

Since the mid 1990's, TSWV has caused severe economic losses on peanut crops in Florida and other parts of the southeastern United States (Culbreath et al., Epidemiology and management of tomato spotted wilt in peanut. Annual Review of Phytopathology 41:53-75 (2003)). Peanut yield losses to spotted wilt can be significant. If 50% of the row feet are showing symptoms of TSWV, research shows that 1,000 to 2,000 pounds of yield per acre will be lost. In addition to this, control measures to reduce crop losses have had significant negative impacts on peanut production. Prior to TSWV, the normal planting density was three to four seeds per foot of row, but to reduce TSWV the planting density has been increased to six to seven seeds per foot of row, effectively doubling seed costs. Similarly, before TSWV the peanut crop was planted during April and early May. To reduce the effects of TSWV, the vast majority of the peanut crop is now planted between May 10^{th} and June 1^{st}. This delayed planting causes conflicts with production of cotton, the major rotational crop with peanut, and shortens the planting window due to constraints of cool weather in the fall months. Because peanut requires an average of 140 days until harvest, later planting reduces yield potential and increases the danger of frost damage in the fall.

Although the date of planting and planting density has been important in reducing losses from spotted wilt, the most effective control measure is cultivar resistance. Thus, a continuing goal of peanut plant breeders is to develop stable, high yielding peanut cultivars that are agronomically sound to maximize the yield produced on the land. To accomplish this goal, the peanut breeder must select and develop peanut plants that have the traits that result in superior cultivars. Several cultivars with moderate resistance to spotted wilt have been developed, but none have sufficient resistance to allow a return to April planting and a planting density of three to four seeds per foot. If the planting window could be returned to April through May, farmers would have significantly more flexibility to plan their operations around weather and cotton production. Therefore, finding new sources and greater levels of resistance to TSWV is highly desirable.

There are numerous steps in the development of any novel, desirable plant germplasm. Plant breeding begins with the analysis, definition of problems and weaknesses of the current germplasm, the establishment of program goals, and the definition of specific breeding objectives. The next step is selection of germplasm that possesses the traits to meet the program goals. The goal is to combine in a single cultivar or hybrid an improved combination of desirable traits from the parental germplasm. These important traits may include improved flavor, higher yield, high oleic acid, improved color, resistance to diseases and insects, tolerance to drought and heat, and better agronomic quality.

Methods for producing novel peanut lines through selection are known in the art. Each of the following references is incorporated in its entirety, herein, by reference: Moore, K. M. et al., J. Heredity 80(3): 252 (1989); Norden, A. J., Peanuts, Culture and Uses. Am. Peanut Res. And Educ. Soc., Stillwater, Okla. (C. T. Wilson ed. 1973); Norden, A. J. in Hybridization of Crop Plants (H. H. Hadley ed. 1980); Norden, A. J., et al., Breeding of the cultivated peanut in Peanut Science and Technology, (H. E. Pattee ed. 1992); Norden, A. J. et al., Florida Agr. Res. 3:16-18 (1984); Knauft, D. A. et al., Peanut, Peanut Principles of Cultivar Development, 2:346-384 (Walter R. Fehr ed. 1987).

### SUMMARY OF THE INVENTION

The present invention provides a novel peanut cultivar designated IPG 913, which is deposited with National Center for Marine Algae and Microbiota International Depository Authority Accession No. 202409005. The invention encompasses the seeds, plants, and plant parts of peanut cultivar IPG 913, as well as plants with essentially all of the physiological and morphological characteristics of IPG 913.

This invention also provides methods for producing a peanut plant by planting a plurality of seeds or by crossing peanut IPG 913 with itself, another peanut line, or a plant of a different species. Any plant breeding methods using peanut cultivar IPG 913 are part of this invention, including selfing, backcrosses, hybrid production, and crosses to populations. All plants and seeds produced using peanut cultivar IPG 913 as a parent are within the scope of this invention, including gene-converted seeds and plants of IPG 913. Methods for introducing a gene conversion, transgene, edited gene, and mutated gene, or gene element, into IPG 913 (i.e., either through traditional breeding, genetic engineering, gene editing, or mutagenesis) are also provided herein.

In still another aspect, the present invention provides regenerable cells for use in cell or tissue culture of peanut plant IPG 913, as well as peanut plants regenerated from these cultures.

### Definitions

To provide a clear and consistent understanding of the specification and claims, the following definitions are provided:

Allele. An allele is any of one or more alternative form of a gene, all of which relate to one trait or characteristic. In a diploid cell or organism, the two alleles of a given gene occupy corresponding loci on a pair of homologous chromosomes.

Alter. The utilization of up-regulation, down-regulation, or gene silencing.

Arachis hypogea L. The domesticated peanut, or groundnut, is an amphidiploid species in the legume or “bean” family and is an annual herbaceous plant.

Backcrossing. A process in which a breeder repeatedly crosses hybrid progeny back to a parental line. For example, a first generation (F₁) hybrid may be crossed with one of the parental lines used to produce the F₁ hybrids to generate a BC₁. Additional generational backcrosses to the recurrent parental line produce BC₂, BC₃, BC₄, BC₅, BC₆, BC₇, BC₈, BC₉, and BC₁₀ generations.

Breeding. The genetic manipulation of living organisms.

Cell. As used herein, this term includes isolated cells, cells grown in tissue culture, and cells that comprise a plant or plant part.

Cotyledon. A cotyledon is a type of seed leaf. The cotyledon contains the food storage tissues of the seed.

Chlorosis. Used to describe a reduced amount of chlorophyll resulting in light or yellow colored leaves.

Concentric chlorotic ring-spots. Light or dark areas on the leaf in the form concentric circles, ovals, or similar shape not necessarily symmetrical or uniform in appearance.

Diploid. A cell or organism having two sets of chromosomes.

Embryo. The plant embryo is the part of a seed or bud that contains the earliest forms of the new plant's roots, stem and leaves.

Essentially all of the physiological and morphological characteristics. A plant having “essentially all the physiological and morphological characteristics” of the cultivar exhibits the characteristics of the cultivar with the exception of any characteristics derived from a converted gene, transgene, edited gene, mutated gene, or somaclonal variant.

F#. Denotes a filial generation, wherein the # is the generation number. For example, F₁ is the first filial generation.

Gene. Refers to a unit of inheritance corresponding to a distinct sequence of DNA or RNA nucleotides that form part of a chromosome. A gene may encode a polypeptide or a nucleic acid molecule that has a function in the cell or organism.

Gene-converted. Describes a plant wherein essentially all of the desired morphological and physiological characteristics of a parental cultivar are maintained with the exception of a single trait that was transferred into the cultivar via breeding (e.g., backcrossing), genetic engineering, gene-editing, or mutagenesis.

Gene mutation. A cell that includes a modified polynucleotide added to or modified within its genome compared to a non-genome mutated cell of the same type. In some cases, a non-genome mutated cell is a wild-type cell. The gene mutation may occur naturally or be induced by the hand of man via (e.g., mutagenesis).

Gene Silencing. The interruption or suppression of the expression of a gene at the level of transcription or translation.

Genetically modified. As used herein, the terms “genetically engineered” and “genetically modified” and are used interchangeably and refer to a prokaryotic or eukaryotic cell whose cellular nucleic acid, whether endogenous and/or exogenous, has been genetically modified or engineered using biotechnology techniques (e.g., transformation, genome editing, RNA interference, gene silencing).

Genome edited. A cell that includes an exogenous, recombinant, synthetic, and/or otherwise modified polynucleotide added to or altered within its genome compared to a non-genome edited cell of the same type. In some cases, a non-genome edited cell is a wild-type cell.

Genotype. Refers to the genetic constitution of a cell or organism.

Habit. This refers to the physical appearance of a plant. In peanuts, it can be prostrate, decumbent, semi-erect, or erect.

Haploid. A cell or organism having a single set of unpaired chromosomes.

Herbicide-tolerant. Used interchangeably with the term “herbicide-resistant” to indicate that a plant or part thereof is capable of growing in the presence of an amount of herbicide that normally causes growth inhibition or phytotoxicity in a non-herbicide-tolerant (e.g., a wild-type) plant or part thereof. Levels of herbicide that normally inhibit growth of a non-tolerant plant are known and readily determined by those skilled in the art. Examples include the quantity of herbicide or rate of application recommended by herbicide manufacturers. The maximum level or rate of herbicide application is the amount of herbicide that would normally inhibit the growth or cause phytotoxicity of a non-herbicide tolerant plant.

Hilum. This refers to the scar left on the seed that marks the place where the seed was attached to the pod prior to the seed being harvested.

Hybrid. Refers to the offspring or progeny of genetically dissimilar plant parents or stock produced as the result of controlled cross-pollination as opposed to a non-hybrid seed produced as the result of natural pollination.

Hypocotyl. A hypocotyl is the portion of an embryo or seedling between the cotyledons and the root. Therefore, it can be considered a transition zone between shoot and root.

LB/A. Pounds per Acre. The seed yield in pounds/acre is the actual yield of the peanut at harvest.

Leaflets. These are part of the plant shoot, and they manufacture food for the plant by the process of photosynthesis.

Leaf petiole. The small stalk attaching the leaf blade to the stem.

Leaf spots. A spot on a leaf usually resultant from infection; can be either chlorotic or necrotic and may be ringed, referred to as a ring-spot.

Linkage. Refers to a phenomenon wherein alleles on the same chromosome tend to segregate together more often than expected by chance if their transmission was independent.

Linkage disequilibrium. Refers to a phenomenon wherein alleles tend to remain together in linkage groups when segregating from parents to offspring, with a greater frequency than expected from their individual frequencies.

Locus. A locus confers one or more traits such as, for example, male sterility, herbicide tolerance, insect resistance, disease resistance, waxy starch, modified fatty acid metabolism, modified phytic acid metabolism, modified carbohydrate metabolism, and modified protein metabolism. The trait may be, for example, conferred by a naturally occurring gene introduced into the genome of the cultivar by backcrossing, a natural or induced mutation, or a transgene introduced through genetic transformation techniques. A locus may comprise one or more alleles integrated at a single chromosomal location.

Maturity Date. Plants are considered mature when 95% of the pods have reached their mature color. The number of days are calculated either from August 31 or from the planting date.

Maturity Group. This refers to an agreed-upon industry division of groups of peanut cultivars based on length of time needed to reach commercial harvest maturity (prior to digging), which is generally considered to be approximately 70% of pods that have black or brown coloration of the mesocarp. These categories can be generally grouped as “early”, reaching harvest maturity by approximately 110-130 days after planting (DAP); “medium”, reaching maturity by approximately 130-145 DAP; and “late”, requiring more than 145 DAP to reach harvest maturity.

Mottling. Abnormal coloration on plants, usually a sign of disease or malnutrition.

Oil or Oil Percent. Peanut seeds contain a considerable amount of oil. Oil is measured by NIR spectrophotometry and is reported as a percentage basis.

Peanut. The seed of a peanut plant, also known as earthnuts, ground nuts, goober peas, monkey nuts, pygmy nuts, and pig nuts.

Peanut flour. Flour high in protein, often used as a gluten-free solution.

Peanut Rx. An index designed to help growers approximate the magnitude of the risk that they face from diseases in the coming season.

Pedigree. Refers to the lineage or genealogical descent of a plant.

Pedigree Distance. Relationship among generations based on their ancestral links as evidenced in pedigrees. May be measured by the distance of the pedigree from a given starting point in the ancestry.

Percent Identity. Percent identity as used herein refers to the comparison of the homozygous alleles of two peanut varieties. Percent identity is determined by comparing a statistically significant number of the homozygous alleles of two developed varieties. For example, a percent identity of 90% between peanut cultivar 1 and peanut cultivar 2 means that the two varieties have the same allele at 90% of their loci.

Percent Similarity. Percent similarity as used herein refers to the comparison of the homozygous alleles of a peanut cultivar such as peanut cultivar IPG 913 with another plant, and if the homozygous allele of peanut cultivar IPG 913 matches at least one of the alleles from the other plant, then they are scored as similar. Percent similarity is determined by comparing a statistically significant number of loci and recording the number of loci with similar alleles as a percentage. A percent similarity of 90% between peanut cultivar IPG 913 and another plant means that peanut cultivar IPG 913 matches at least one of the alleles of the other plant at 90% of the loci.

Plant. As used herein, the term “plant” includes plant cells, plant protoplasts, and plant cell tissue cultures from which peanut plants can be regenerated; plant calli, plant clumps, meristematic cells, and plant cells that are intact in plants; and parts of plants, such as embryos, pollen, ovules, seeds, flowers, glumes, panicles, leaves, stems, shoots, suckers, internodes, buds, roots, root tips, anthers, and pistils.

Plant Height. Plant height is taken from the top of the soil to the top node of the plant and is measured in centimeters.

Plant parts. Includes, without limitation, protoplasts, leaves, stems, internodes, buds, roots, root tips, anthers, pistils, seed, grain, nut, peanut, embryo, pollen, ovules, cotyledon, hypocotyl, pod, flower, shoot, tissue, petiole, pedicel, pistils, cells, meristematic cells, and the like.

Pod. This refers to the fruit of a peanut plant. It consists of the hull or shell (pericarp) and the peanut seeds.

Progeny. As used herein, includes an F₁ peanut plant produced from the cross of two peanut plants where at least one plant includes peanut cultivar IPG 913 and progeny further includes, but is not limited to, subsequent generations.

Protein Percent. Peanut seeds contain a considerable amount of protein. Protein is generally measured by NIR spectrophotometry and is reported as percentage basis.

Quantitative Trait Loci. Quantitative Trait Loci (QTL) refers to genetic loci that control to some degree, numerically representable traits that are usually continuously distributed.

Regeneration. Regeneration refers to the development of a plant from tissue culture.

Resistance. The intrinsic ability of a plant to tolerate or withstand external stimuli or pressure (e.g., pressure from diseases, insects, herbicides, heat, cold, drought, salinity, abiotic, and biotic stressors).

Seed Oleic Acid Content. The percentage of oleic acid in the peanut, as measured by gas chromatography or near-infrared spectrometry. High seed oleic acid content is equal to a range of approximately 70-80%. Normal seed oleic acid content is equal to a range of approximately 40-50%.

Seeds. Includes seeds and plant propagules of all kinds including, but not limited to, true seeds, seed pieces, suckers, corms, bulbs, fruit, tubers, grains, nuts, peanuts, cuttings, cut shoots and the like. However, in preferred embodiments, it refers to true seeds.

Single gene converted. Single gene converted or conversion plant refers to plants which are developed by traditional breeding methods (e.g., backcrossing), via genetic engineering, gene-editing, or mutagenesis, wherein essentially all of the desired morphological and physiological characteristics of a line are recovered in addition to the single gene transferred into the line via the breeding technique, genetic engineering, gene-editing, or mutagenesis.

Subline. Although peanut cultivar IPG 913 contains substantially fixed genetics, is phenotypically uniform with no off-types expected, there still remains a small proportion of segregating loci either within individuals or within the population as a whole. A breeder of ordinary skill in the art may fix these loci by making them more uniform in order to optimize the performance of the cultivar. One example of this type of approach is described in the “breeding bias” methods described in U.S. Pat. No. 5,437,697 and may be utilized by a breeder of ordinary skill in the art to further purify the cultivar in order to increase its yield. By sublining in this manner, no crosses to a different cultivar are made, and so a new genetic cultivar is not created and the overall genetic composition of the cultivar remains essentially the same.

Thrips. Tiny insects which carry and transmit disease. The two most common species being Tobacco thrips (Frankliniella fusca) and Western flower thrips (Frankliniella occidentalis). They are also referred to as thunderflies, thunderbugs, storm flies, thunderblights, and corn lice.

Tomato Spotted Wilt Tospovirus (TSWV). A class V virus having a single stranded RNA genome with negative polarity found within the family Bunyaviridae. TSWV is an arbovirus usually vectored by thrips and is common in warm climates such as Asia, America, Europe and Africa.

Total Sound Mature Kernels (TSMK). The percentage of SMK (sound mature kernels) riding a screen with 0.64 by 1.91 cm slots plus sound splits (i.e. the sum of SMK and sound splits). TSMK is the commercially-standardized metric for evaluating grade or quality of peanuts and thereby directly impacts sale-ability.

Trait. Refers to an observable and/or measurable characteristic of an organism.

Transgene. Any DNA sequences, whether from a different species or from the same species, which are introduced into the genome using transformation or various breeding methods are referred to herein collectively as “transgenes.”

Variety. Synonymous with cultivar, a substantially homozygous peanut line which may comprise minor modifications thereof, including but not limited to resulting from sublining, creation of a doubled haploid line, a locus conversion, a mutation, a transgene, an edited gene, or a somaclonal variant, but which otherwise retains the overall genetics of the peanut line.

Wild-type. When made in reference to a gene, “wild-type” refers to a functional gene common throughout a plant population and, thus, arbitrarily designated the “normal” or “wild-type” form of the gene.

Yield (Pounds/Acre). The yield in pounds/acre is the actual yield of the peanut at harvest.

### DETAILED DESCRIPTION OF THE INVENTION

The present invention provides a novel peanut cultivar designated IPG 913, which is deposited with National Center for Marine Algae and Microbiota International Depository Authority Accession No. 202409005. The invention encompasses both the seeds of this cultivar and plants grown from these seeds. The invention further encompasses any peanut plant having all or essentially all of the physiological and morphological characteristics peanut cultivar IPG 913.

As used herein, the term plant includes plant cells, plant protoplasts, plant cell tissue cultures from which peanut plants can be regenerated, plant calli, plant clumps, and parts of plants, such as leaves, stems, internodes, buds, roots, root tips, anthers, pistils, seed, nut, peanut, embryo, pollen, ovules, cotyledon, hypocotyl, pod, flower, shoot, tissue, petiole, pedicel, pistil, cells, meristematic cells, and the like.

### Development and Characterization of Peanut Cultivar IPG 913 (Experimental Number ‘19-RS1-0913’)

Peanut cultivar IPG 913 is a normal-oleic runner-type cultivar with moderate resistance to spotted wilt caused by Tomato Spotted Wilt Virus. Additionally, Peanut cultivar IPG 913 has large seed that place it in the upper range of runner-type cultivars. Peanut cultivar IPG 913 has an excellent proportion of total sound mature kernels (TSMK). Peanut cultivar IPG 913 has very high yield potential when compared to cultivars of similar maturity especially in the presence of spotted wilt and has excellent agronomic characteristics including a prominent mainstem and runner growth (prostrate) habit.

Some of the selection criteria used in developing IPG 913 include the following traits: pod yield, grade, seed size, fatty acid composition, oil content, oleic acid content, disease resistance, seedling emergence, disease tolerance, TSWV resistance, herbicide tolerance, maturity, and late season plant intactness. In addition, the cultivar has been phenotypically selected upon for uniformity of plant type, pod type, and stability, as described in the following cultivar description information. Off-type plants (for either plant architecture or pod type or other phenotypic variation) have been rogued at various stages of cultivar development, and the cultivar has been increased by self-pollination with continued observation of and selection for uniformity.

Table 1 describes the developmental timeline for cultivar IPG 913, while Table 2 outlines the phenotypic characteristics of cultivar IPG 913. The results of 2020-2024 yield trials for cultivar IPG 913 and commercial checks are presented in Tables 3-19. Stability and uniformity observations by year and location are reported for cultivar IPG 913 in Table 20.

IPG 913 outyielded all 6 commercial check cultivars in Early County, GA in 2021 (Table 3); 4 out of 5 commercial checks in Tifton, GA in 2022 (Table 6); all 3 commercial checks in Headland, AL in 2022 (Table 8); all 5 and all 7 commercial cultivars in Terrell County, GA in 2022 and 2023, respectively (Tables 4 and 10); and all 3 commercial checks in Fairhope, AL in 2023 (Table 12). Additionally, IPG 913 exhibited a lower percentage incidence of TSWV, a lower percentage of stunted plants, and higher percentage of TSMK than several commercial checks in the aforementioned trials.

TABLE 1
IPG 913 development timeline

| Year | Program stage |
|---|---|
| 2024 | Yield Trials: Tifton, Georgia (Tables 14, 17 & 18); Terrell Co., Georgia (Table |
| | 15); Fairhope, Alabama (Tables 16 & 19). |
| 2023 | Terrell County, Georgia Yield Trials (Tables 9, 10 & 13); Tifton, Georgia |
| | Yield Trial (Table 11); Fairhope, Alabama Yield Trial (Table 12). The Terrell |
| | County, Georgia Yield Trials were evaluated under elevated TSWV pressure. |
| 2022-2023 | Juana Diaz, Puerto Rico Winter Nursery Increase (Table 14) |
| 2022 | Terrell County, Georgia Yield Trial (Table 4 & 5); Tifton, Georgia Yield Trial |
| | (Tables 6 & 7); Headland, Alabama Yield Trial (Table 8). The Terrell County, |
| | Georgia Yield Trials were evaluated under elevated TSWV pressure. |
| 2021-2022 | Juana Diaz, Puerto Rico Winter Nursery Increase (Table 14) |
| 2021 | Early County, Georgia Yield Trial (Table 3). The Early County, Georgia Yield |
| | Trials were evaluated under elevated TSWV pressure. |
| 2020 | Terry County, Texas Yield Trial (Table 14) |
| 2019 | 396 M4: 8 plants were planted in two-row plots, and seed from a single selected |
| | plant was saved to plant the following year. IPG 913 (experimental number ‘19- |
| | RS1-0913’) was selected as a uniform line and bulk-harvested for replicated |
| | yield testing in 2020. |
| 2018 | 519 M4: 7 plants were planted in two-row plots, and seed from a single selected |
| | plant was saved to plant the following year. |
| 2017 | 538 M4: 6 plants were planted in two-row plots, and seed from a single selected |
| | plant was saved to plant the following year. |
| 2016 | 223 M4: 5 plants were planted in rows, and seed from a single selected plant |
| | was saved to plant the following year. |
| 2015 | In 2015, M4 families were evaluated in the field and were selected for a number |
| | of different desirable characteristics. Approximately 223 individual plants were |
| | selected, hand-harvested, and replanted (plant-to-row) in 2016, per the common |
| | procedure for pedigree plant breeding. |
| 2014 | M3 families were planted in the field for self-pollination and subsequent seed |
| | increase. All viable M3 plants that produced seed were bulk-harvested. |
| 2013 | M2 families were planted in the field for self-pollination and subsequent seed |
| | increase. All viable M2 plants that produced seed were bulk-harvested. |
| 2012 | Cultivar ‘ACI 149’ (U.S. PVP 201200372) was chemically mutagenized to |
| | produce novel, segregating mutant populations. These populations were |
| | planted in the field for self-pollination and subsequent seed increase in 2012. |
| | All viable M1 plants that produced seed were bulk-harvested within each |
| | respective segregating mutant population for propagation in 2013. |

Peanut cultivar IPG 913 is similar to peanut cultivar ACI 149 with similar vegetative growth habits, leaf color, and seed shape. However, cultivar IPG 913 exhibits several distinct phenotypic differences compared to ACI 149, including but not limited to: 1) plant size: IPG 913 has both a taller mainstem and longer lateral branches than ACI 149, but ACI 149 has larger leaflets (in both length and width) than IPG 913; 2) seed size: seed of IPG 913 has an overall larger seed size (in both length and width) than ACI 149, resulting in a greater proportion of runner Jumbo kernels and a lesser proportion of Medium kernels compared to ACI 149; 3) pod yield: IPG 913 pod yield is notably greater than that of ACI 149; 4) TSMK; IPG 913 has a greater percentage of total sound mature kernels (TSMK) than ACI 149; 5) Tomato spotted wilt virus resistance: IPG 913 exhibits greater resistance to TSWV than ACI 149; and 6) Oleic acid content: IPG 913 is a normal-oleic cultivar, while ACI 149 is a high-oleic cultivar.

Peanut cultivar IPG 913 has similar maturity to peanut cultivar Georgia-06G, both having 140 days to maturity (Table 2). However, IPG 913 exhibits several phenotypic differences compared to Georgia-06G, including: 1) whole peanut kernel size: whole peanut kernels of IPG 913 are larger than those of Georgia-06G (Tables 3, 5, and 7); and 2) TSWV resistance: IPG 913 exhibited a high level of resistance to spotted wilt and infection by TSWV in most trials, while Georgia-06G exhibited moderate tolerance to spotted wilt and was more susceptible to infection by TSWV (Tables 4, 6, 8, 11, 14-15, and 18-19).

Peanut cultivar IPG 913 is similar to peanut cultivar Georgia-16HO with some phenotypic differences, including: 1) fatty acid composition: IPG 913 has normal seed oleic acid content whereas Georgia-16HO has high seed oleic acid content; and 2) TSWV resistance: IPG 913 has a high level of resistance to spotted wilt and infection by TSWV, while Georgia-16HO has moderate tolerance to spotted wilt and is more susceptible to infection by TSWV (Tables 4, 6, 9, 11, 14-15, and 19).

TABLE 2
IPG 913 cultivar description information.

| Category | Parameter | Description |
|---|---|---|
| Plant | Growth habit: | Prostrate |
| | Flowering on the | None |
| | Mainstem: | |
| | Branching pattern: | Alternate |
| | Branching: | Profuse |
| | Mainstem height: | 42 cm |
| Maturity | Region: | Georgia, United States |
| | Number of days to | 140 |
| | maturity: | |
| | Days earlier than | 19 |
| | comparison peanut | |
| | cultivar Georgia-12Y: | |
| | Days later than | 0 |
| | comparison peanut | |
| | cultivar Georgia-06G: | |
| Leaves | Arrangement: | Opposite, pinnate, |
| | | and tetrafoliate |
| | Leaflet length: | 4.0 cm |
| | Leaflet width: | 1.6 cm |
| | Leaflet length/width ratio: | 2.5 |
| | Leaflet color (Munsell): | 7.5GY 3/4 |
| Flower | Color: | Yellow |
| | Days to flowering: | 25 to 35; indeterminate |
| | Arrangement: | Axillary; from leaf axil |
| Pod | Shape: | Oblong, indehiscent legume |
| | Length: | 29 mm |
| | Diameter: | 15 mm |
| | Number of seeds per pod: | 2 |
| | Pod yield (lb/A): | 3000 to 7000 |
| | Surface: | Glabrous |
| | Constriction: | Medium |
| | Beak: | Inconspicuous |
| Seed | Coat color: | Light tan |
| | Coat surface: | Smooth |
| | Shape: | Cylindrical Blunt Ends |
| | Grams per 100 seeds | 79.4 |
| | (8% moisture): | |
| | Length: | 15 mm |
| | Width: | 10 mm |

TABLE 3
Final pod yield, grade, and seed size distribution results of cultivar
IPG 913 and six commercial cultivars in Early County, Georgia in 2021.

| | | | | | | | Seed Oleic |
|---|---|---|---|---|---|---|---|
| | Pod Yield | TSWV^{a} | TSMK^{b} | Jumbo^{c} | Medium^{d} | No. 1^{e} | Acid |
| Entry | lb/A | % | | | | | Content^{f} |
| IPG 913 | 4055 | 8 | 62 | 42 | 35 | 13 | Normal |
| Georgia-16HO | 2932 | 9 | 62 | 31 | 37 | 15 | High |
| TUFRunner | 2325 | 14 | 53 | 31 | 36 | 16 | High |
| 727 | | | | | | | |
| Georgia-06G | 2234 | 9 | 66 | 38 | 37 | 13 | Normal |
| Georgia-09B | 2223 | 19 | 62 | 28 | 37 | 14 | High |
| TUFRunner | 1752 | 36 | 59 | 25 | 39 | 18 | High |
| 297 | | | | | | | |
| IPG QR-14 | 1100 | 30 | 56 | 9 | 39 | 27 | High |

^{a}Tomato spotted wilt virus.
^{b}Total sound mature kernels; a combination of whole peanut kernels that did not pass through a 5.9-mm × 19.0-mm screen and sound splits that did not pass through a 6.7-mm round screen.
^{c}Whole peanut kernels that did not pass through a 8.3-mm × 19.0-mm screen.
^{d}Whole peanut kernels that did not pass through a 7.1-mm × 19.0-mm screen.
^{e}Whole peanut kernels that did not pass through a 5.9-mm × 19.0-mm screen.
^{f}Seed oleic acid content, as measured by gas chromatography or near-infrared spectrometry, where “Normal” values range between 40-50%, and “High” values range between 70-80%.

TABLE 4
Pod yield, incidence and severity of tomato spotted wilt virus, late
leaf spot ratings, and seed oleic acid results of cultivar IPG 913
and five commercial cultivars in Terrell County, Georgia in 2022.

| | | TSWV^{a} | | Late Leaf Spot | | |
|---|---|---|---|---|---|---|
| | Pod Yield | Incidence^{b} | Severity | 109 DAP^{d} | 130 DAP | Total |
| Entry | lb/A | % | 1-9^{c} | 1-10^{f} | | AUDPC^{e} |
| IPG 913 | 3988 | 14 | 3.2 | 1.8 | 3.5 | 2.7 |
| Georgia-06G | 3301 | 19 | 3.7 | 2.0 | 2.6 | 2.3 |
| Georgia-09B | 3263 | 25 | 4.7 | 2.5 | 3.2 | 2.9 |
| AU-NPL 17 | 3237 | 4 | 3.5 | 2.3 | 2.8 | 2.6 |
| Georgia-16HO | 2769 | 23 | 4.0 | 2.5 | 4.2 | 3.4 |
| TUFRunner ™ | 2238 | 20 | 5.1 | 2.5 | 4.2 | 3.4 |
| ‘727’ | | | | | | |

^{a}Tomato spotted wilt virus.
^{b}Incidence ratings defined by percentage conducted by Dr. Albert Culbreath, research plant pathologist with the Univ. of Georgia on 8 Sep. 2022 (120 DAP).
^{c}Visual rating of TSWV severity for each plot; “1” = no disease symptoms and “9” = plant necrosis; ratings conducted by Dr. Dylan Wann of IPG on 28 Aug. 2022 (109 DAP).
^{d}Days after planting (11 May 2022).
^{e}Florida 1-10 rating scale; 1 = 0% defoliation and 10 = 100% defoliation.
^{f}Area under the disease progress curve (late leaf spot).

TABLE 5
Pod yield, grade, and seed size distribution results of cultivar IPG
913 and five commercial cultivars in Terrell County, Georgia in 2022.

| | | | | | | Seed Oleic |
|---|---|---|---|---|---|---|
| | Pod Yield | TSMK^{a} | Jumbo^{b} | Medium^{c} | No. 1^{d} | Acid |
| Entry | lb/A | % | | | | Content^{e} |
| IPG 913 | 3988 | 72 | 78 | 1.0 | 7 | Normal |
| Georgia-06G | 3301 | 71 | 75 | 3.0 | 7 | Normal |
| Georgia-09B | 3263 | 70 | 77 | 1.5 | 8 | High |
| AU-NPL 17 | 3237 | 69 | 76 | 2.0 | 7 | High |
| Georgia-16HO | 2769 | 69 | 71 | 2.0 | 8 | High |
| TUFRunner ™ | 2238 | 64 | 68 | 2.5 | 10 | High |
| ‘727’ | | | | | | |

Means within a column followed by the same lowercase letter are not significantly different according to Fisher's Least Significant Difference test at P = 0.05.
^{a}Total sound mature kernels; a combination of whole peanut kernels that did not pass through a 5.9-mm × 19.0-mm screen and sound splits that did not pass through a 6.7-mm round screen.
^{b}Whole peanut kernels that did not pass through a 8.3-mm × 19.0-mm sizing screen.
^{c}Whole peanut kernels that did not pass through a 7.1-mm × 19.0-mm sizing screen.
^{d}Whole peanut kernels that did not pass through a 6.3-mm × 19.0-mm sizing screen.
^{e}Seed oleic acid content, as measured by gas chromatography or near-infrared spectrometry, where “Normal” values range between 40-50%, and “High” values range between 70-80%.

TABLE 6
Pod yield, tomato spotted wilt virus, late leaf spot, and
emergent plant stand results of cultivar IPG 913 and four
commercial cultivars near Tifton, Georgia in 2022.

| | Pod | | Late Leaf | Emergent |
|---|---|---|---|---|
| | Yield | TSWV^{b} | Spot | Plant Stand^{d} |
| Entry | lb/A | % | 1-10^{c} | no./row foot |
| FloRun ™ ‘331’ | 5905 | 18 | 6.5 | 4.5 |
| IPG 913 | 5724 | 17 | 6.2 | 4.7 |
| Georgia-16HO | 5641 | 19 | 7.0 | 5.3 |
| AU NPL-17 | 5321 | 17 | 6.0 | 4.4 |
| Georgia-06G | 5226 | 23 | 7.2 | 3.7 |

^{a} Total sound mature kernels; a combination of whole peanut kernels that did not pass through a 5.9-mm × 19.0-mm screen and sound splits that did not pass through a 6.7-mm round screen.
^{b}Tomato spotted wilt virus as measured by percentage incidence.
^{c}Florida 1-10 leaf spot rating scale; 1 = no disease and 10 = plants defoliated or dead.
^{d}Stand counts measured in number of plants per row foot conducted on 16 May 2022 (18 Days After Pollination).

TABLE 7
Pod yield, grade, seed size distribution, and seed oleic acid content of cultivar
IPG 913 and four commercial cultivars near Tifton, Georgia in 2022.

| | | | | | | Seed Oleic |
|---|---|---|---|---|---|---|
| | Pod Yield^{a} | TSMK^{b} | Jumbo^{c} | Medium^{d} | No. 1^{e} | Acid |
| Entry | lb/A | % | | | | Content^{e} |
| FloRun ™ ‘331’ | 5905 | 74 | 82 | 0.5 | 7.0 | High |
| IPG 913 | 5724 | 74 | 88 | 0.5 | 5.5 | Normal |
| Georgia-16HO | 5641 | 73 | 88 | 0.5 | 5.5 | High |
| AU-NPL 17 | 5321 | 70 | 88 | 1.0 | 4.5 | High |
| Georgia-06G | 5226 | 74 | 85 | 1.0 | 6.0 | Normal |

^{a}Total sound mature kernels; a combination of whole peanut kernels that did not pass through a 5.9-mm × 19.0-mm screen and sound splits that did not pass through a 6.7-mm round screen.
^{b}Whole peanut kernels that did not pass through a 8.3-mm × 19.0-mm sizing screen.
^{c}Whole peanut kernels that did not pass through a 7.1-mm × 19.0-mm sizing screen.
^{d}Whole peanut kernels that did not pass through a 6.3-mm × 19.0-mm sizing screen.
^{e}Seed oleic acid content, as measured by gas chromatography or near-infrared spectrometry, where “Normal” values range between 40-50%, and “High” values range between 70-80%.

TABLE 8
Pod yield, emergent plant stand, severity of Tomato spotted
wilt virus, and seed oleic acid results of cultivar IPG 913
and three commercial cultivars in Headland, Alabama in 2022.

| | | | | | Emergent | |
|---|---|---|---|---|---|---|
| | | | | Late | Plant | |
| | | | | Leaf | Stand^{d} | Seed Oleic |
| | Pod Yield | TSMK^{a} | TSWV^{b} | Spot | no./row | Acid |
| Entry | lb/A | % | | 1-10^{c} | foot | Content^{e} |
| IPG 913 | 5130 | 76 | 17 | 3.7 | 4.6 | Normal |
| Georgia-16HO | 4991 | 73 | 13 | 3.8 | 4.7 | High |
| Georgia-06G | 4392 | 74 | 17 | 3.8 | 4.9 | Normal |
| FloRun ™ ‘331’ | 3534 | 80 | 29 | 4.1 | 4.1 | High |

^{a}Total sound mature kernels; a combination of whole peanut kernels that did not pass through a 5.9-mm × 19.0-mm screen and sound splits that did not pass through a 6.7-mm round screen, reported in percentages.
^{b}Tomato spotted wilt virus; incidence ratings reported in percentages and conducted by Dr. Amanda Strayer-Scherer, extension plant pathologist with Auburn Univ. on 29 Jul. 2022.
^{c}Florida 1-10 leaf spot rating scale; 1 = no disease and 10 = plants defoliated or dead; incidence ratings conducted by Dr. Amanda Strayer-Scherer, extension plant pathologist with Auburn Univ. on 27 Sep. 2022.
^{d}Stand counts measured in number of plants per row foot conducted on 6 Jun. 2022 (28 DAP).
^{e}Seed oleic acid content, as measured by gas chromatography or near-infrared spectrometry, where “Normal” values range between 40-50%, and “High” values range between 70-80%.

TABLE 9
Pod yield, commercial grade and seed size distribution, and
seed oleic acid content results of cultivar IPG 913 and seven
commercial cultivars in Terrell County, Georgia in 2023.

| | | | | | | Seed Oleic |
|---|---|---|---|---|---|---|
| | Pod Yield | TSMK^{a} | Jumbo^{b} | Medium^{c} | No. 1^{d} | Acid |
| Entry | lb/A | % | | | | Content^{e} |
| ACI 147 | 2839 | 74 | 19 | 52 | 12 | High |
| ACI 149 | 2890 | 74 | 40 | 31 | 10 | High |
| AU-NPL 17 | 4575 | 74 | 65 | 17 | 5 | High |
| IPG 913 | 4705 | 80 | 63 | 17 | 5 | Normal |
| Georgia-06G | 4681 | 78 | 60 | 19 | 4 | Normal |
| Georgia-09B | 4409 | 78 | 49 | 24 | 7 | High |
| Georgia-12Y | 4202 | 74 | 53 | 32 | 5 | Normal |
| Georgia-16HO | 4314 | 78 | 55 | 18 | 6 | High |

^{a}Total sound mature kernels; a combination of whole peanut kernels that did not pass through a 5.9-mm × 19.0-mm screen and sound splits that did not pass through a 6.7-mm round screen.
^{b}Whole peanut kernels that did not pass through a 8.3-mm × 19.0-mm sizing screen.
^{c}Whole peanut kernels that did not pass through a 7.1-mm × 19.0-mm sizing screen.
^{d}Whole peanut kernels that did not pass through a 6.3-mm × 19.0-mm sizing screen.
^{e}Seed oleic acid content, as measured by gas chromatography or near-infrared spectrometry, where “Normal” values range between 40-50%, and “High” values range between 70-80%.

TABLE 10
Pod yield, incidence of tomato spotted wilt virus, plant stand,
and seed oleic acid results of cultivar IPG 913 and seven
commercial cultivars in Terrell County, Georgia in 2023.

| | | | | Harvest | |
|---|---|---|---|---|---|
| | Pod | TSWV^{a} | | Plant | Seed Oleic |
| | Yield | Incidence^{b} | Severity | Stand^{d} | Acid |
| Entry | lb/A | % | 1-9^{c} | no./row foot | Content^{e} |
| IPG 913 | 4705 | 16 | 3.8 | 4.1 | Normal |
| Georgia-06G | 4681 | 15 | 3.7 | 4.4 | Normal |
| AU-NPL 17 | 4575 | 15 | 3.7 | 4.0 | High |
| Georgia-09B | 4409 | 19 | 4.2 | 4.2 | High |
| Georgia-16HO | 4314 | 24 | 4.3 | 4.2 | High |
| Georgia-12Y | 4202 | 10 | 2.5 | 4.1 | Normal |
| ACI 149 | 2890 | 23 | 3.9 | 3.4 | High |
| ACI 147 | 2839 | 53 | 6.5 | 3.9 | High |

^{a}Tomato spotted wilt virus.
^{b}Incidence ratings reported in percentages and conducted by Dr. Dylan Wann of IPG on 22 Aug. 2023 (106 DAP).
^{c}Visual rating of TSWV severity for each plot; “1” = no disease symptoms and “9” = plant necrosis; ratings conducted by Dr. Dylan Wann of IPG on 22 Aug. 2023 (106 DAP).
^{d}Stand counts measured in number of plants per row foot and conducted immediately after digging on 26 Sep. 2023 (141 DAP).
^{e}Seed oleic acid content, as measured gas chromatography or near-infrared spectrometry, where “Normal” values range between 40-50%, and “High” values range between 70-80%.

TABLE 11
Pod yield and tomato spotted wilt incidence and
severity results of cultivar IPG 913 and four commercial
cultivars near Tifton, Georgia in 2023.

TSWV^{a}

| | | |
|---|---|---|
| Pod | | Stunted |
| Yield | Incidence^{b} | Plants^{c} |

| | | |
|---|---|---|
| Entry | lb/A | -----------------%---------------- |

| | | | |
|---|---|---|---|
| Georgia-12Y | 5959 | 10 | 9 |
| AU-NPL 17 | 5938 | 23 | 17 |
| Georgia-06G | 5559 | 26 | 24 |
| IPG 913 | 5558 | 21 | 18 |
| Georgia-16HO | 5147 | 25 | 24 |

^{a}Tomato spotted wilt virus.
^{b}Incidence ratings reported in percentages.
^{c}Stunted plants ratings reported in percentages

TABLE 12
Pod yield, emergent plant stand, incidence and severity of tomato
spotted wilt virus, and seed oleic acid results of cultivar IPG
913 and three commercial cultivars in Fairhope, Alabama in 2023.

| | | Emergent | | | |
|---|---|---|---|---|---|
| | Pod | Plant | TSWV^{b} | | Seed Oleic |
| | Yield | Stand^{a} | Incidence^{c} | Severity^{d} | Acid |
| Entry | lb/A | no./row foot | % | 1-9 | Content^{e} |
| IPG 913 | 4975 | 2.5 | 6 | 1.3 | Normal |
| Georgia-06G | 4126 | 2.7 | 16 | 3.8 | Normal |
| AU-NPL 17 | 4092 | 2.8 | 9 | 2.7 | High |
| Georgia-16HO | 4000 | 2.9 | 18 | 4.0 | High |

^{a}Emergent stand counts measured in number of plants per row foot conducted on 30 May 2023 (14 DAP).
^{b}Tomato spotted wilt virus; disease ratings conducted by Dr. Dylan Wann of IPG on 20 Aug. 2023 (96 DAP).
^{c}Incidence ratings reported in percentages.
^{d}Visual rating of TSWV severity for each plot; “1” = no disease symptoms and “9” = plant necrosis.
^{e}Seed oleic acid content, as measured by gas chromatography or near-infrared spectrometry, where “Normal” values range between 40-50%, and “High” values range between 70-80%.

TABLE 13
Leaflet length and width, mainstem height, lateral branch length, and leaflet color of
cultivar IPG 913 and seven commercial cultivars in Terrell County, Georgia in 2023.

| | | | | Lateral | |
|---|---|---|---|---|---|
| | Leaflet | Leaflet | Mainstem | Branch | |
| | Length^{a} | Width^{a} | Height | Length | Leaflet |
| Entry | -----------------------------------cm----------------------------------- | | | | Color^{a, b} |
| ACI 147 | 4.1 | 1.7 | 31 | 37 | 7.5GY 4/4 |
| ACI 149 | 4.2 | 1.7 | 35 | 42 | 7.5GY 3/4 |
| AU-NPL 17 | 4.7 | 1.9 | 41 | 46 | 7.5GY 3/4 |
| IPG 913 | 4.0 | 1.6 | 39 | 45 | 7.5GY 3/4 |
| Georgia-06G | 3.8 | 1.5 | 39 | 41 | 7.5GY 4/4 |
| Georgia-09B | 4.1 | 1.8 | 38 | 48 | 5GY 3/4 |
| Georgia-12Y | 3.9 | 1.6 | 39 | 48 | 7.5GY 3/4 |
| Georgia-16HO | 3.9 | 1.4 | 35 | 42 | 7.5GY 4/4 |

^{a}Leaflet data were collected from the basal leaflet of the first fully-formed leaf at the top of the mainstem and are reported in centimeters.
^{b}Color determined using the Munsell Plant Tissue Color Book (2012).

TABLE 14
Pod yield; incidence of tomato spotted wilt virus, late
leaf spot, and white mold; emergent plant stand; and
seed oleic acid results of one advanced runner peanut
line and five cultivars near Tifton, Georgia in 2024.

| | | | Late | Emergent | |
|---|---|---|---|---|---|
| | Pod | | Leaf | Plant | Seed Oleic |
| | Yield^{a} | TSWV^{b} | Spot | Stand^{d} | Acid |
| | lb/A | % | 1-10^{c} | no/row foot | Content |
| Georgia-06G | 5944 | 15 | 4.1 | 3.1 | Normal |
| Georgia-12Y | 5944 | 3 | 3.3 | 4.3 | Normal |
| AU-NPL 17 | 5919 | 11 | 3.6 | 3.6 | High |
| IPG 913 | 5795 | 6 | 4.3 | 2.7 | Normal |
| Georgia-16HO | 5770 | 14 | 5.8 | 3.9 | High |
| Georgia-22MPR | 5358 | 4 | 5.0 | 2.9 | High |

^{a}Standardized at 10% moisture.
^{b}Tomato spotted wilt virus; ratings conducted at 87 days after planting.
^{c}Florida 1-10 leaf spot visual rating scale; 1 = no disease and 10 = plants defoliated or dead; ratings were conducted at 87 days after planting.
^{d}Stand counts were conducted at 18 days after planting.

TABLE 15
Pod yield; incidence of tomato spotted wilt virus, late leaf spot, and white
mold; emergent plant stand; and seed oleic acid results of one advanced
runner peanut line and five cultivars in Terrell Co., Georgia in 2024.

| | | | | | Emergent | |
|---|---|---|---|---|---|---|
| | | | | | Plant | Seed |
| | | | Late Leaf | White | Stand^{e} | Oleic |
| | Pod Yield^{a} | TSWV^{b} | Spot | Mold^{d} | no./row | Acid |
| Entry | lb/A | % | 1-10^{c} | % | foot | Content |
| Georgia-12Y | 5620 | 10 | 5.0 | 4 | 5.0 | Normal |
| Georgia-06G | 5194 | 12 | 6.3 | 10 | 3.9 | Normal |
| IPG 913 | 5140 | 8 | 5.5 | 3 | 3.7 | Normal |
| Georgia- | 4990 | 10 | 7.7 | 3 | 4.2 | High |
| 16HO | | | | | | |
| AU-NPL 17 | 4961 | 12 | 6.2 | 5 | 4.4 | High |
| ACI 149 | 3247 | 46 | 7.0 | 21 | 4.7 | High |

^{a}Standardized at 10% moisture.
^{b}Tomato spotted wilt virus; ratings conducted by Dr. Robert Kemerait of the Univ. of Georgia at 120 days after planting.
^{c}Florida 1-10 leaf spot visual rating scale; 1 = no disease and 10 = plants defoliated or dead; ratings were conducted at 145 days after planting by Dr. Dylan Wann of IPG.
^{d}White mold ratings conducted immediately after digging at 146 days after planting by Dr.
Dylan Wann of IPG.
^{e}Stand counts conducted at 15 days after planting.

TABLE 16
Pod yield; incidence of tomato spotted wilt virus, late leaf spot, and white
mold; emergent plant stand; and seed oleic acid results of one advanced
runner peanut line and three cultivars near Fairhope, Alabama in 2024.

| | | | | | Emergent | Seed |
|---|---|---|---|---|---|---|
| | | | Late Leaf | White | Plant Stand^{e} | Oleic |
| | Pod Yield^{a} | TSWV^{b} | Spot | Mold^{d} | no./row | Acid |
| Entry | lb/A | % | 1-10^{c} | % | foot | Content |
| Georgia- | 6540 | 0 | 3.1 | 1 | 3.2 | High |
| 16HO | | | | | | |
| Georgia-12Y | 5696 | 0 | 3.0 | 1 | 3.4 | Normal |
| IPG 913 | 5588 | 1 | 3.1 | 2 | 3.3 | Normal |
| AU-NPL 17 | 5293 | 0 | 3.1 | 1 | 3.4 | High |
| Georgia-06G | 5155 | 0 | 2.8 | 3 | 3.2 | Normal |

^{a}Standardized at 10% moisture.
^{b}Tomato spotted wilt virus; ratings conducted by Dr. Amanda Scherer of Auburn Univ. at 55 days after planting.
^{c}Florida 1-10 leaf spot visual rating scale; 1 = no disease and 10 = plants defoliated or dead; ratings were conducted at 136 days after planting by Dr. Amanda Scherer of Auburn Univ.
^{d}White mold ratings conducted at 136 days after planting by Dr. Amanda Scherer of Auburn Univ.
^{e}Stand counts conducted at 14 days after planting.

TABLE 17
Pod yield, tomato spotted wilt virus and late leaf spot
incidence, and emergent plant stand results of one advanced
peanut line and seven cultivars within three different
digging date treatments near Tifton, Georgia in 2024.

| | | | | Late | Emergent |
|---|---|---|---|---|---|
| | | Pod | | Leaf | Plant |
| Digging | | Yield^{b} | TSWV^{c} | Spot | Stand^{e} |
| Date | Entry | lb/A | % | 1-10^{d} | no./row foot |
| 135 DAP^{a} | | | | | |
| | IPG 913 | 6134 | 6 | 2.0 | 3.1 |
| | Arnie | 5799 | 4 | 2.7 | 2.4 |
| | FloRun-52N | 5685 | 4 | 3.0 | 3.7 |
| | Georgia-12Y | 5610 | 6 | 2.9 | 3.8 |
| | TifNV-HG | 5571 | 10 | 3.7 | 3.3 |
| | Georgia-06G | 5504 | 9 | 3.0 | 3.7 |
| | Georgia-22MPR | 5411 | 6 | 3.4 | 2.0 |
| | Georgia-21GR | 5384 | 9 | 3.0 | 2.3 |
| 145 DAP | | | | | |
| | IPG 913 | 6758 | 13 | 5.0 | 3.1 |
| | TifNV-HG | 6455 | 11 | 4.1 | 3.5 |
| | FloRun-52N | 6361 | 6 | 3.9 | 3.5 |
| | Arnie | 6241 | 6 | 3.7 | 2.8 |
| | Georgia-06G | 5952 | 8 | 4.5 | 3.5 |
| | Georgia-22MPR | 5867 | 10 | 4.5 | 2.1 |
| | Georgia-12Y | 5625 | 8 | 3.8 | 3.5 |
| | Georgia-21GR | 5598 | 9 | 3.6 | 2.3 |
| 155 DAP | | | | | |
| | TifNV-HG | 7127 | 14 | 5.3 | 3.2 |
| | Arnie | 6689 | 9 | 4.8 | 2.5 |
| | Georgia-12Y | 6622 | 12 | 5.0 | 3.6 |
| | Georgia-21GR | 6120 | 10 | 5.0 | 2.2 |
| | Georgia-06G | 6006 | 11 | 5.5 | 4.1 |
| | IPG 913 | 5996 | 15 | 6.0 | 2.9 |
| | FloRun-52N | 5919 | 9 | 4.8 | 3.5 |
| | Georgia-22MPR | 5069 | 13 | 5.6 | 2.3 |

^{a}Days after planting.
^{b}Standardized at 10% moisture.
^{c}Tomato spotted wilt virus.
^{d}Florida 1-10 leaf spot visual rating scale; 1 = no disease and 10 = plants defoliated or dead.
^{e}Stand counts conducted at 14 DAP.

TABLE 18
Factorial pod yield, tomato spotted wilt virus and late leaf
spot incidence, and emergent plant stand results of one
advanced peanut line and seven cultivars and three different
digging date treatments near Tifton, Georgia in 2024.

| | | | | Late | Emergent |
|---|---|---|---|---|---|
| | Digging | Pod | | Leaf | Plant |
| | Date | Yield^{b} | TSWV^{c} | Spot | Stand^{e} |
| Entry | DAP^{a} | lb/A | % | 1-10^{d} | no./row foot |
| TifNV-HG | 155 | 7127 | 14 | 5.3 | 3.2 |
| IPG 913 | 145 | 6758 | 13 | 5.0 | 3.1 |
| Arnie | 155 | 6689 | 9 | 4.8 | 2.5 |
| Georgia-12Y | 155 | 6622 | 12 | 5.0 | 3.6 |
| TifNV-HG | 145 | 6455 | 11 | 4.1 | 3.5 |
| FloRun-52N | 145 | 6361 | 6 | 3.9 | 3.5 |
| Arnie | 145 | 6241 | 6 | 3.7 | 2.8 |
| IPG 913 | 135 | 6134 | 6 | 2.6 | 3.1 |
| Georgia-21GR | 155 | 6120 | 10 | 5.0 | 2.2 |
| Georgia-06G | 155 | 6006 | 11 | 5.5 | 4.1 |
| IPG 913 | 155 | 5996 | 15 | 6.0 | 2.9 |
| Georgia-06G | 145 | 5952 | 8 | 4.5 | 3.5 |
| FloRun-52N | 155 | 5919 | 9 | 4.8 | 3.5 |
| Georgia-22MPR | 145 | 5867 | 10 | 4.5 | 2.1 |
| Arnie | 135 | 5799 | 4 | 2.7 | 2.4 |
| FloRun-52N | 135 | 5685 | 4 | 3.0 | 3.7 |
| Georgia-12Y | 145 | 5625 | 8 | 3.8 | 3.5 |
| Georgia-12Y | 135 | 5610 | 6 | 2.9 | 3.8 |
| Georgia-21GR | 145 | 5598 | 9 | 3.6 | 2.3 |
| TifNV-HG | 135 | 5571 | 10 | 3.7 | 3.3 |
| Georgia-06G | 135 | 5504 | 9 | 3.0 | 3.7 |
| Georgia-22MPR | 135 | 5411 | 6 | 3.4 | 2.0 |
| Georgia-21GR | 135 | 5384 | 9 | 3.0 | 2.3 |
| Georgia-22MPR | 155 | 5069 | 13 | 5.6 | 2.3 |

^{a}Days after planting.
^{b}Standardized at 10% moisture.
^{c}Tomato spotted wilt virus.
^{d}Florida 1-10 leaf spot visual rating scale; 1 = no disease and 10 = plants defoliated or dead.
^{e}Stand counts conducted at 14 DAP.

TABLE 19
Pod yield and tomato spotted wilt virus, late leaf spot,
and white mold incidence results of one advanced peanut
line and nineteen cultivars in Fairhope, Alabama in 2024.

| | | | Late | |
|---|---|---|---|---|
| | Pod | | Leaf | White |
| | Yield | TSWV^{a} | Spot | Mold |
| Entry | lb/A | % | 1-10^{b} | % |
| IPG 913 | 5726 | 0 | 2.1 | 4 |
| TifNV-High O/L | 5552 | 0 | 2.2 | 4 |
| FloRun 331 | 5479 | 1 | 2.2 | 1 |
| FloRun-52N | 5444 | 0 | 2.2 | 3 |
| TifNV-HG | 5390 | 0 | 2.2 | 3 |
| Georgia-16HO | 5373 | 1 | 2.5 | 4 |
| Georgia-18RU | 5322 | 1 | 2.1 | 3 |
| FloRun-T61 | 5309 | 0 | 5.4 | 2 |
| Georgia-19HP | 5279 | 1 | 2.8 | 2 |
| Georgia-22MPR | 5212 | 0 | 4.1 | 3 |
| TifCB-7 | 5126 | 0 | 2.5 | 3 |
| Georgia-12Y | 5066 | 0 | 2.7 | 1 |
| Georgia-06G | 4890 | 1 | 2.5 | 2 |
| ACI 222 | 4834 | 3 | 4.6 | 2 |
| ACI 3321 | 4818 | 0 | 2.1 | 2 |
| Georgia-20VHO | 4818 | 0 | 2.1 | 2 |
| Arnie | 4724 | 1 | 3.0 | 3 |
| ACI 212 | 4722 | 1 | 4.6 | 3 |
| Georgia-21GR | 4672 | 2 | 2.8 | 3 |
| AU-NPL 17 | 4644 | 1 | 4.5 | 2 |

All disease ratings conducted by Dr. Amanda Scherer of Auburn Univ.
^{a}Tomato spotted wilt virus.
^{b}Florida 1-10 leaf spot visual rating scale; 1 = no disease and 10 = plants defoliated or dead.

TABLE 20
Crop years and locations of production and observation
of cultivar IPG 913 for stability and uniformity.

| Crop Year | Test Location | Plant Type^{a} | Pod Type^{b} |
|---|---|---|---|
| 2020 | Terry Co., TX | Uniform | Uniform |
| 2021 | Early Co., GA | Uniform | Uniform |
| 2021-2022 | Juana Diaz, PR | Uniform | Uniform |
| 2022 | Headland, AL; Terrell Co., GA; | Uniform | Uniform |
| | Tifton, GA; Terry Co., TX | | |
| 2022-2023 | Juana Diaz, PR | Uniform | Uniform |
| 2023 | Early Co., GA; Fairhope, AL; Headland, | Uniform | Uniform |
| | AL; Jackson Co., MS; Mississippi Co., | | |
| | AR; Portageville, MO; Terrell Co., GA; | | |
| | Tifton, GA; | | |
| 2024 | Beaumont, MS; Brewton, AL; Fairhope, | Uniform | Uniform |
| | AL; Headland, AL; Jackson Co., MS; Live | | |
| | Oak, FL; Marianna, FL; Midville, GA; | | |
| | Mississippi Co., AR; Portageville, MO; | | |
| | Raymond, MS; Shorter, AL; Stoneville, | | |
| | MS; Terrell Co., GA; Terry Co., TX; | | |
| | Tifton, GA; Verona, MS | | |

^{a}Plant type is determined based on phenotypic observation of plant architecture. A “Uniform” rating indicates the absence of obvious off-type plants.
^{b}Pod type is determined based on phenotypic observation of pod size and shape. A “Uniform” rating indicates the absence of obvious off-type pods.

### Methods

This present invention provides methods for producing peanut plants. In some embodiments, these methods involve crossing a first parent peanut plant with a second parent peanut plant wherein either the first or second parent peanut plant is a peanut plant of the cultivar IPG 913. Further, both first and second parent peanut plants may be peanut cultivar IPG 913. Self-pollinated plants of peanut cultivar IPG 913 are part of the invention, including repeated generations of self-pollinated plants of the invention or creation of doubled haploid plants of the invention. Still further, this invention also is directed to methods for producing a peanut cultivar IPG 913-derived peanut plant by crossing peanut cultivar IPG 913 with a second peanut plant and growing the progeny seed, wherein the crossing and growing steps may be repeated with the peanut cultivar IPG 913-derived plant from 0 to 7 times, or more. Thus, any such methods using the peanut cultivar IPG 913 are part of this invention: selfing, recurrent selection, pedigree breeding, backcrosses, hybrid production, crosses to populations, and the like. All plants produced using peanut cultivar IPG 913 as a parent are within the scope of this invention, including plants derived from peanut cultivar IPG 913. Advantageously, the peanut cultivar is used in crosses with different peanut cultivars to produce first generation (F₁) peanut seeds and plants with superior characteristics.

In one aspect, a IPG 913-derived peanut plant, a progeny plant, a genetically modified plant, a transformed plant, a mutated plant, a gene-edited plant, a regenerated plant, somaclonal variant, or other genetic variant is selected that has molecular markers, morphological characteristics, and/or physiological characteristics in common with IPG 913 (e.g., those listed in Table 2).

Particular markers used for these purposes are not limited to any particular set of markers, but are envisioned to include any type of marker and marker profile which provides a means of distinguishing varieties for identification or selection purposes. Primers and PCR protocols for assaying these and other markers may be used for identification of peanut cultivar IPG 913, and plant parts and plant cells of peanut cultivar IPG 913. The genetic profile (i.e., genotype) may be used to identify a peanut plant produced through the use of peanut cultivar IPG 913; or to verify a pedigree for progeny plants or derivative plants produced through the use of peanut cultivar IPG 913. The genetic marker profile is also useful in breeding and developing backcross conversions. For example, a plant of cultivar IPG 913 comprising a single gene conversion, transgene, modified gene, edited gene, or genetic sterility factor, may be identified by having a molecular marker profile with a high percent identity to peanut cultivar IPG 913. Such a percent identity might be 95%, 96%, 97%, 98%, 99%, 99.5%, or 99.9% identical to peanut cultivar IPG 913. The genetic marker profile during conversion or modification may also be ascertained for purposes of recovering a higher percentage of the recurrent parent genome (i.e., during backcrossing) via measuring either percent identity or percent similarity.

Examples of molecular markers include: Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs) (which are also referred to as Microsatellites), and Single Nucleotide Polymorphisms (SNPs).

In another aspect, IPG 913 may be self-pollinated or subjected to the process of creating doubled haploids, the processes of which fix or make homozygous residual heterozygous alleles at one or more loci in the IPG 913 genome. The resulting plants have all or essentially all of the physiological and morphological characteristics of IPG 913.

Further, this invention provides methods for introducing a desired trait into peanut cultivar IPG 913. This may be accomplished using traditional breeding methods, such as backcrossing (see Breeding Methods section below). Alternatively, the desired trait may be introduced by transforming the peanut cultivar with a transgene (see Transformation Methods section below), by mutagenizing a gene within the peanut's genome (see Mutagenesis Methods section below), or by editing a gene within the peanut's genome (see Gene Editing Methods section below). The transgenic, mutant, or edited cultivar produced by these methods may be crossed via traditional breeding techniques with another cultivar to produce a new transgenic, mutant, or edited cultivar. Alternatively, a transgene, mutated gene, or edited gene could be moved into cultivar IPG 913 using traditional breeding techniques, transformation, or gene-editing methods.

Optionally, any of the disclosed methods may further comprise additional steps involving producing peanut seed from the resulting peanut plants and/or planting the peanut seed.

Genetic modifications conferring desirable traits are produced using several methods that are known in the art, including, without limitation, the introduction of polymorphisms, deletions, insertions, mutated genes, converted genes, edited genes, exogenous DNA, and exogenous DNA comprising a native gene or gene element. The genetic modification functions to silence, repress, reduce, or increase the expression of a native gene; or to modify the product produced by a native gene.

The present invention encompasses all plants, or parts thereof, produced by the methods described herein, as well as the seeds produced by these plants. Further, any plants derived from peanut cultivar IPG 913 or produced from a cross using cultivar IPG 913 are provided, including crosses with IPG 913 as a parent to produce F₁ seeds, and the resulting F₁ plants produced by growing said seeds. The invention also relates to a plant of peanut cultivar IPG 913 comprising a genetic variant, including somaclomal variants, produced through the following methods, without limitation: traditional breeding methods, transformation, mutagenesis, or gene-editing, as well as plants produced in a male-sterile form. Notably, this includes gene-converted or trait-converted plants developed via any of these methods. For example, IPG 913 may be used as a recurrent parent in backcross-breeding to add a desired trait or gene from a donor parent to produce higher generation backcross plants otherwise having all or essentially all of the physiological and morphological characteristics of IPG 913, as determined at a 5% significance level when grown under the same environment. The desired trait or gene may be derived from a native trait or gene, a product of genetic engineering (i.e., gene-edited or transformed), or a mutation created by mutagenesis. Thus, the invention relates to plants derived from IPG 913 or variants of IPG 913, but otherwise which have all or essentially all of the physiological and morphological characteristics of IPG 913.

The present invention also encompasses progeny of peanut cultivar IPG 913 comprising a combination of at least two IPG 913 traits selected from those listed in the Tables and Detailed Description of the Invention, wherein the progeny peanut plant is not significantly different from IPG 913 for said traits, as determined at the 5% significance level when grown in the same environment. One of skill in the art knows how to compare a trait between two plant varieties to determine if there is a significant difference between them (Fehr and Walt, Principles of Cultivar Development, pp. 261-286 (1987)). Molecular markers or mean trait values may be used to identify a plant as progeny of IPG 913. Alternatively, progeny may be identified through their filial relationship with peanut cultivar IPG 913 (e.g., as being within a certain number of breeding crosses of peanut cultivar IPG 913). For example, progeny produced by the methods described herein may be within 1, 2, 3, 4, 5, or more breeding crosses of peanut cultivar IPG 913.

Traits of agronomic and/or economic interest include, without limitation: herbicide resistance; insect resistance; resistance to bacterial, fungal, or viral disease; modified fatty acid metabolism; modified carbohydrate metabolism; modified seed yield; yield stability; stress resistance; modified protein percent; modified fancy pod percent; modified pod size, shape, or color; maturity; and male sterility.

The specific gene(s) conferring a trait of interest may be any known in the art or listed herein, including: a polynucleotide conferring resistance to imidazolinone, dicamba, sulfonylurea, glyphosate, glufosinate, triazine, benzonitrile, cyclohexanedione, phenoxy proprionic acid, and L-phosphinothricin; a polynucleotide encoding a Bacillus thuringiensis polypeptide; a polynucleotide encoding phytase, FAD-2, FAD-3, galactinol synthase, or a raffinose synthetic enzyme; or a polynucleotide conferring resistance to rust (Puccinia arachidis), early and late leaf spot (Cercospora arachidicola and Cercosporidium personatum), web blotch (Didymella arachidicola), pepper spot (Leptosphaerulina crassiasca), Tomato Spotted Wilt Tospovirus (TSWV), atmospheric scorch, chemical burn, iron chlorosis, potato leafhopper (Empoasca fabae) seedling disease (Rhizoctonia solani, Pythium spp., Fusarium spp. and others), yellow mold (Aspergillus flavus, Aspergillus parasiticus), root knot nematode (Meloidogyne arenaria) root lesion nematode, southern blight (Sclerotium rolfsii), Sclerotinia blight (Sclerotinia minor), Rhizoctonia pod, peg and limb rot (Rhizoctonia solani), pythium pod rot (Pythium myriotylum), botrytis blight (Botrytis cinerea), black mold (Aspergillus niger), blackhull (Thielaviopsis basicola), Phymatotrichum root rot (Phymatotrichum omnivorum), and tooth fungus (Phanerochaeta sp).

Any of the seeds, plants, or plant parts provided may be utilized for human food, livestock feed, and as a raw material in industry (see Industrial Uses section below). The present invention also encompasses methods of producing a commodity plant product. Exemplary commodity plant products that can be produced from peanut cultivar IPG 913 include, but are not limited to, edible oil, peanut butter, roasted nuts, salted nuts, livestock feed, flour, soaps, and plastics.

### Tissue Culture

The present invention provides tissue cultures of regenerable cells or protoplasts produced from peanut cultivar IPG 913. As is well known in the art, tissue culture of peanut can be used for the in vitro regeneration of a peanut plant. Thus, such cells and protoplasts may be used to produce plants having the physiological and morphological characteristics of peanut cultivar IPG 913. The peanut plants regenerated by these methods are also encompassed by the present invention.

As used herein, the term “tissue culture” describes a composition comprising isolated cells or a collection of such cells organized into parts of a plant. Exemplary tissues for tissue or cell culture include protoplasts, calli, plant clumps, meristematic cells, and plant cells. Examples of additional plant parts that may be used for tissue or cell culture include embryos, pollen, ovules, hypocotyls, cotyledons, seeds, flowers, glumes, panicles, leaves, stems, shoots, suckers, internodes, buds, roots, root tips, anthers, pedicels, petioles, and pistils. Culture of various peanut cells or tissues and regeneration of plants therefrom is well known in the art.

Methods for culturing plant tissues are known in the art. General descriptions of such methods are provided, for example, by Maki, et al., “Procedures for Introducing Foreign DNA into Plants” in Methods in Plant Molecular Biology & Biotechnology, Glick, et al., (Eds. pp. 67-88 CRC Press, 1993); and by Phillips, et al., “Cell-Tissue Culture and In-Vitro Manipulation” in Corn & Corn Improvement, 3rd Edition; Sprague, et al., (Eds. pp. 345-387 American Society of Agronomy Inc., 1988).

### Breeding Methods

The goal of peanut breeding is to develop new, superior peanut cultivars and hybrids. A superior cultivar is produced when a new combination of desirable traits is formed within a single plant cultivar. Desirable traits may include, but are not limited to, those listed in the Methods section. Single genes may be transferred into the line via the breeding.

The breeding methods used with the present invention may involve a single-seed descent procedure, in which one seed per plant is harvested and used to plant the next generation. Alternatively, the methods may utilize a multiple-seed procedure, in which one or more seeds harvested from each plant in a population is threshed together to form a bulk which is used to plant the next generation.

Use of peanut cultivar IPG 913 in any plant breeding method is encompassed by the present invention. The choice of a breeding or selection method will depend on several factors, including the mode of plant reproduction, the heritability of the trait(s) being improved, and the type of cultivar used commercially (e.g., F₁ hybrid cultivar, pureline cultivar). Popular selection methods include pedigree selection, modified pedigree selection, mass selection, recurrent selection, backcrossing, or a combination thereof.

Pedigree selection is commonly used for the improvement of self-pollinating crops. Two parents are crossed to produce an F₁ population. An F₂ population is produced by selfing one or several F₁ plants. Selection of the best individuals may begin in the F₂ population; then, beginning in the F₃ generation, the best individuals in the best families are selected. Replicative testing of families can begin in the F₄ generation to make selection of traits with low heritability more effective. At an advanced stage of inbreeding (e.g., F₆ or F₇), the best lines are tested for potential release as new cultivars.

Mass and recurrent selections can be used to improve populations of either self- or cross-pollinating crops. A genetically variable population of heterozygous individuals is either identified or created by intercrossing several different parents. A genetically variable population may also be created by subjecting a cultivar to mutagenesis. The best plants within the genetically variable population are selected based on individual superiority, outstanding progeny, or excellent combining ability. The selected plants are intercrossed to produce a new population, which often undergoes additional cycles of selection.

Backcrossing is commonly used to transfer genes for highly heritable traits into a desirable homozygous cultivar or variety. The term “backcrossing” refers to the repeated crossing of hybrid progeny back to one of the parental plants, referred to as the recurrent parent. The plant that serves as the source of the transferred trait is called the donor parent. After the initial cross, individuals possessing the transferred trait are selected and repeatedly crossed to the recurrent parent to produce higher filial generation backcross plants. The resulting plant is expected to have the attributes of the recurrent parent along with the trait transferred from the donor parent. Molecular markers may be used to determine the percentage of donor and recurrent parent genome at each stage of backcrossing, and plants with higher percentages of recurrent parent markers may be selected to recover a higher percentage of recurrent parent while also introgressing the desired trait from the donor parent. Phenotypic traits may also be measured and selected upon, or optionally in combination with molecular markers, to recover recurrent parent traits. The resulting higher filial generation backcross progeny plants will otherwise have all or essentially all of the physiological and morphological characteristics of IPG 913, as determined at a 5% significance level when grown under the same environment.

### Methods of Genetically Modifying Plants

The present invention also encompasses methods of genetically modifying plants of peanut cultivar ‘IPG 913’ to produce peanut varieties comprising essentially all of the physiological and morphological characteristics of ‘IPG 913’ but comprising at least one new trait. Methods of producing a genetically modified peanut plants may rely on any of the recombinant DNA methodology or other methods know to those of skill in the art. For example, plants may be genetically modified by transformation, mutagenesis (including chemical mutagenesis or transposon mutagenesis), genome editing (such as CRISPR/Cas based genome editing or Cre/loxP or other recombinase-based modification) or using RNA interference or gene silencing (via knocking out genes or RNA-based silencing) to genetically modify a peanut plant, or a cell thereof to prepare a genetically modified peanut plant. DNA sequences native to peanut, as well as non-native DN A sequences, can be transformed into peanut and used to alter levels of native or non-native proteins. Various promoters, targeting sequences, enhancing sequences, and other DNA sequences can be inserted into the genome for the purpose of altering the expression of proteins.

The plants may be modified and selected for a wide range of agronomic, physiologic, morphologic or other traits. Traits that may be genetically modified include, but are not limited to, increasing herbicide resistance; insect resistance; or resistance to bacterial, fungal, or viral disease. The plants may also be modified to have modified fatty acid metabolism; modified carbohydrate metabolism; modified seed yield; yield stability; stress resistance; modified protein percent; modified fancy pod percent; modified pod size, shape, or color; or male sterility. These traits may be conferred by increasing or decreasing expression of one or more genes. Insect resistance may include resistance to an insect selected from thrips, southern corn rootworm, burrowing bug, lesser cornstalk borer, leaf hopper, aphid and nematode. Tomato spotted wilt virus and other diseases are transferred to plants by insects. For example, TSWV is transferred most commonly by Tobacco thrips (Frankliniella fusca) and Western flower thrips (Frankliniella occidentalis). The disease resistance may be selected from southern stem rot, late leaf spot, cylindrocladium black rot, Sclerotinia blight, early leaf spot, tomato spotted wilt virus and pod rot complex. The modified fatty acid content may be altered concentrations or relative concentrations of oleic acid, linoleic acid and palmitic acid in the peanuts produced by the plants.

Also encompassed herein are methods of introducing the TSWV resistance trait of ‘IPG 913’ into another peanut variety. This can be accomplished via conventional breeding methods by crossing the ‘IPG 913’ peanut with anther peanut cultivar that lacks adequate resistance to TSWV and selecting for progeny plants with increased resistance to TSWV. The selected progeny plants can then be crossed to either parent to produce new progeny and further selected for resistance to TSWV. Further backcrossing can be completed to obtain the TSWV resistant progeny.

### Transformation Methods

As is noted above, the present invention provides plants and seeds of peanut cultivar IPG 913 in which additional traits have been transferred. While such traits may be selected for using traditional breeding methods, they may also be introduced as transgenes. “Transgenes” include both foreign genes and additional or modified versions of native genes. Plants can be genetically engineered to have a wide variety of traits of agronomic interest. Desirable traits may include without limitation those listed in the Methods section.

Alternatively, transgenic peanut plants in which a gene is silenced (e.g., via knockout, antisense technology, co-suppression; RNA interference, virus-induced gene silencing, target-RNA-specific ribozymes, hairpin structures, microRNA, and ribozymes) or transgenic peanut plants that express a foreign protein for commercial production may be generated using peanut cultivar IPG 913.

Transgenes are typically introduced in the form of an expression vector. As used herein, an “expression vector” is DNA comprising a gene operatively linked to a regulatory element (e.g., a promoter). The expression vector may contain one or more such gene/regulatory element combinations. The expression vector may also include additional sequences, such as a signal sequence or a tag, that modify the protein produced by the transgene. The vector may be a plasmid and can be used alone or in combination with other plasmids.

Expression vectors include at least one genetic marker operably linked to a regulatory element (e.g., a promoter) that allows transformed cells containing the vector to be recovered by selection. In some embodiments, negative selection, i.e., inhibiting growth of cells that do not contain the selectable marker gene, is utilized. Negative selection markers include, for example, genes that result in detoxification of a chemical agent (e.g., an antibiotic or an herbicide) and genes that result in insensitivity to an inhibitor. Exemplary negative selection genes include neomycin phosphotransferase II (nptII), hygromycin phosphotransferase, gentamycin acetyl transferase, streptomycin phosphotransferase, and aminoglycoside-3′-adenyl transferase. In other embodiments, positive selection, i.e., screening for the product encoded by a reporter gene, is utilized. Exemplary reporter genes include β-glucuronidase, β-galactosidase, luciferase, chloramphenicol acetyltransferase, and Green Fluorescent Protein (GFP).

Transgene expression is typically driven by operably linking the transgene to a promoter within the expression vector. However, other regulatory elements may also be used to drive expression, either alone or in combination with a promoter. As used herein, a “promoter” is a region of DNA upstream of a transcription start site that is involved in recognition and binding of RNA polymerase for transcription initiation. Any class of promoter may be selected to drive the expression of a transgene. For example, the promoter may be “tissue-specific”, “cell type-specific”, “inducible”, or “constitutive”. Those of skill in the art know how to select a suitable promoter based the particular circumstances and genetic engineering goals.

Methods for producing transgenic plants are well known in the art. General descriptions of plant expression vectors, reporter genes, and transformation protocols can be found in Gruber, et al., “Vectors for Plant Transformation”, in Methods in Plant Molecular Biology & Biotechnology in Glick, et al., (Eds. pp. 89-119, CRC Press, 1993). Methods of introducing expression vectors into plant tissue include direct gene transfer methods, such as microprojectile-mediated delivery, DNA injection, and electroporation, as well as the direct infection, or co-cultivation of plant cells with Agrobacterium tumefaciens, described for example by Horsch et al., Science, 227:1229 (1985). Descriptions of Agrobacterium vector systems and methods for Agrobacterium-mediated gene transfer are provided by Gruber, et al., supra.

In addition, transgenes created in other peanut plants may be transferred in to peanut cultivar IPG 913 using breeding methods (e.g., backcrossing), genetic engineering (e.g., transformation), or via gene editing (e.g., CRISPR-mediated homology-directed repair).

### Mutagenesis Methods

Mutagenesis is another method of introducing new traits into peanut cultivar IPG 913. The goal of artificial mutagenesis is to increase the rate of mutation for a desired characteristic or trait. Desirable traits may include without limitation those listed in the Methods section. Mutation rates can be increased by many different means including temperature, long-term seed storage, tissue culture conditions, radiation (e.g., X-rays, Gamma rays, neutrons, Beta radiation, or ultraviolet radiation), or chemical mutagens (e.g., base analogues such as 5-bromo-uracil or diethyl sulfate), related compounds (e.g., 8-ethoxy caffeine), antibiotics (e.g., streptonigrin), alkylating agents (e.g., sulfur mustards, nitrogen mustards, epoxides, ethylenamines, sulfates, sulfonates, sulfones, lactones), azide, hydroxylamine, nitrous acid, and acridines. Once a desired trait is generated through mutagenesis, the trait may then be incorporated into existing germplasm by traditional breeding techniques (e.g., backcrossing). Details of mutation breeding can be found in Fehr, “Principles of Cultivar Development,” Macmillan Publishing Company (1993).

In addition, mutations, including single mutated genes, created in other peanut plants may be transferred into peanut cultivar IPG 913 via genetic engineering (e.g., transformation) or gene editing (e.g., CRISPR-mediated homology-directed repair). The resulting backcrossed, mutated, transformed, or gene-edited plants will otherwise have all or essentially all of the physiological and morphological characteristics of IPG 913, as determined at the 5% significance level when grown under the same environment.

### Gene Editing Methods

In some embodiments, new traits are introduced into peanut cultivar IPG 913 via CRISPR-mediated homology-directed repair. Desirable traits may include without limitation those listed in the Methods section. “Homology directed repair (HDR)” is a naturally occurring nucleic acid repair system that is initiated by the presence of double strand breaks (DSBs) in DNA. In CRISPR-mediated HDR, CRISPR is used to create targeted DSBs (i.e., by targeting a nuclease to cut at specific loci using guide RNAs that are complementary to those loci), which are then repaired using a donor template. The donor template comprises a sequence for insertion flanked by segments of DNA that are homologous to the ends of the DSBs. Thus, in cells that repair the DSBs using the donor template, the genome will be edited to include the sequence for insertion between the sites of the DSBs. Any form of donor template known in the art may be used in the methods of the present invention, including single-stranded oligodeoxynucleotides (ssODNs) and donor plasmids. The nuclease may be naturally existing or engineered. Examples of nucleases include meganucleases, zinc finger nucleases (ZFN), transcription activator-like effector nucleases (TALENs), and the Cas9-guideRNA system (adapted from CRISPR).

In addition, edited genes created in other peanut plants may be transferred into peanut cultivar IPG 913 using breeding methods (e.g., backcrossing), genetic engineering (e.g., transformation), or gene-editing (e.g., CRISPR-mediated homology-directed repair). The resulting backcrossed, mutated, transformed, or gene-edited plants will otherwise have all or essentially all of the physiological and morphological characteristics of IPG 913, as determined at a 5% significance level when grown under the same environment.

### INDUSTRIAL USES

The TSWV resistance trait can be introgressed into other varieties in the runner-type market class (A. hypogaea subsp. hypogaea var. hypogaea botanical type Virginia) as well as the Virginia (A. hypogaea subsp. hypogaea var. hypogaea botanical type Virginia), Peruvian (A. hypogaea subsp. hypogaea var. hypogaea botanical type Peruvian runner), Valencia (A. hypogaea subsp. fastigata var. fastigata botanical type Valencia), and Spanish (A. hypogaea subsp. fastigata var. vulgaris botanical type Spanish) market classes. Peanuts in the runner-type market class are the most commonly used varieties and are found in diverse products such as peanut butter, salted nuts and confectionery products. On the other hand, peanut varieties in the Virginia market class are largely used as salted nuts and in-shell market. The Valencia is largely used in peanut butter while the Spanish type is used in certain niche markets where small round peanuts are needed such as confectionery products and red skin peanuts. Finally, the Peruvian runner market class is grown in certain regions of Mexico.

Peanut is recognized as one of the major oilseed crops and as a rich source of protein. In the United States peanuts are primarily utilized as whole seeds for human foods such as peanut butter, roasted seeds, and confections. In recent years the United States has been the leading exporter of peanuts for human consumption; peanuts rank ninth in area among the row crops and second in dollar value per acre. Peanuts are rich in nutrients, providing over 30 essential nutrients and phytonutrients, and are a good source of niacin, folate, fiber, magnesium, vitamin E, manganese and phosphorus. They are also naturally free of trans-fats and sodium, and contain about 25% protein. Because of these qualities, organizations like the World Health Organization, UNICEF, Project Peanut Butter and Doctors Without Borders have used peanut products to help save malnourished children in developing countries. Thus, improvement of the factors that indicate and/or affect both the food quality of peanuts and the peanut harvest is of considerable importance to the worldwide peanut processing and manufacturing community.

All publications cited in this application are herein incorporated by reference. The foregoing examples of the related art and limitations related therewith are intended to be illustrative and not exclusive. Other limitations of the related art will become apparent to those of skill in the art upon a reading of the specification.

### Deposit Information

A deposit of the peanut cultivar IPG 913 disclosed above and recited in the appended claims has been made with the Provasoli-Guillard National Center for Marine Algae and Microbiota (NCMA) (60 Bigelow Drive, East Boothbay, ME 04544). The date of deposit was Sep. 12, 2024. The deposit of 625 seeds was taken from the same deposit maintained by International Peanut Group (1995#B County Road 290, Brownfield, Texas 79316) since prior to the filing date of this application. All restrictions will be irrevocably removed upon granting of a patent, and the deposit is intended to meet all of the requirements of 37 C.F.R. §§ 1.801-1.809. The Accession Number provided by the International Depositary Authority is NCMA 202409005. The deposit will be maintained in the depository for a period of thirty years, or five years after the last request, or for the enforceable life of the patent, whichever is longer, and will be replaced as necessary during that period.

What is claimed is:

1. A seed of Arachis Hypogaea L. peanut cultivar designated ‘IPG 913’, a representative sample of seed of said cultivar having been deposited under National Center for Marine Algae and Microbiota International Depositary Authority Accession No. 202409005.

2. A peanut plant, or a part thereof, produced by growing the seed of claim 1.

3. A method for producing peanut plants, said method comprising planting a plurality of peanut seeds as recited in claim 1 under conditions favorable for the growth of peanut plants, and allowing the peanut seeds to grow into peanut plants.

4. The method of claim 3, further comprising the step of producing peanut seeds from the resulting peanut plants.

5. A peanut seed produced by the method of claim 4.

6. A tissue culture of regenerable cells or protoplasts produced from the peanut plant of claim 2.

7. A peanut plant regenerated from the tissue culture of claim 6, wherein the regenerated peanut plant has all the morphological and physiological characteristics of ‘IPG 913’, as determined at a 5% significance level when grown under the same environmental conditions.

8. A method for producing an F₁ hybrid peanut seed, wherein the method comprises crossing a first parent peanut plant with a second parent peanut plant to produce an F₁ hybrid peanut seed, wherein the first parent peanut plant is the peanut plant of claim 2, and the second parent peanut plant is a peanut cultivar, and allowing peanut seeds to develop.

9. An F₁ hybrid peanut seed produced by the method of claim 8.

10. The method of claim 8, further comprising the step of producing an F₁ hybrid peanut plant by planting the F₁ hybrid peanut seed under conditions favorable for the growth of peanut plants to produce an F₁ hybrid peanut plant. The method of claim 8, wherein at least one of the first parent peanut plant or second parent peanut plant is genetically modified.

11. A method of producing a genetically modified peanut plant comprising transforming, mutating, genome editing, or using RNA interference or gene silencing to genetically modify the peanut plant of claim 2, or a cell thereof, to produce a genetically modified peanut plant, wherein the genetically modified peanut plant has all the morphological and physiological characteristics of ‘IPG 913’, as determined at a 5% significance level when grown under the same environmental conditions.

12. The method of claim 11, wherein the genetically modified peanut plant is modified for a trait selected from the group consisting of herbicide resistance, insect resistance, bacterial resistance, fungal resistance, viral resistance, fatty acid metabolism, carbohydrate metabolism, seed yield, yield stability, stress resistance, protein percent, fancy pod percent, pod size, pod shape, pod color; and male sterility.

13. The method of claim 12, wherein the trait is increased Tomato Spotted Wilt Virus (TSWV) resistance.

14. A peanut plant or part thereof, or peanut seed, produced by the method of claim 11.

15. A method of introducing a desired trait into peanut cultivar ‘IPG 913’ said method comprising the steps of:
a. crossing the plant as recited in claim 2 with a plant of another peanut line expressing the desired trait, to produce F₁ hybrid peanut seeds;
b. planting the F₁ hybrid peanut seeds under conditions favorable for the growth of peanut plants to produce F₁ hybrid peanut plants;
c. selecting F₁ hybrid peanut plants that express the desired trait, to produce selected F₁ hybrid peanut plants;
d. crossing the selected F₁ hybrid peanut plants with the plant of claim 2 to produce BC₁ peanut seeds;
e. planting the BC₁ peanut seeds under conditions favorable for the growth of peanut plants to produce BC₁ peanut plants;
f. selecting the BC₁ peanut plants that express both the desired trait and some or all of the physiological and morphological characteristics of peanut cultivar ‘IPG 913’, to produce selected BC₁ peanut plants; and
g. backcrossing the selected BC₁ peanut plants three or more times in succession to produce selected higher filial generation backcross plants that express both the desired trait and the physiological and morphological characteristics of peanut cultivar ‘IPG 913’, as determined at a 5% significance level when grown under the same environmental conditions when grown in the same environmental conditions.

16. The method of claim 15, additionally comprising the step of planting a plurality of peanut seeds produced by the selected higher filial generation backcross plants under conditions favorable for the growth of peanut plants and optionally comprising the step of producing peanut seeds from the resulting peanut plants.

17. The peanut plants, or parts thereof, resulting from growing the peanut seeds of claim 16, wherein the peanut plants express the desired trait, and wherein the peanut plants otherwise comprise all of the morphological and physiological characteristics of peanut cultivar ‘IPG 913’, as determined at a 5% significance level when grown under the same environmental conditions.

18. A method of producing a commodity plant product, said method comprising obtaining the seed of claim 1 or a part thereof, and producing a commodity plant product therefrom.

19. The method of claim 18, wherein the commodity plant product is selected from the group consisting of edible oil, peanut butter, roasted nuts, salted nuts, raw nuts, confectionary products, flour, livestock feed, biodiesel, fuel, mulch, manufacturing particle board, soaps, fertilizer and plastics.

20. A method of introducing the tomato spotted wilt virus (TSWV) resistance trait of an Arachis Hypogaea L. peanut cultivar designated ‘IPG 913’ into another peanut cultivar, the method comprising:
a. crossing the ‘IPG 913’ plant with a plant of a second peanut line that is not resistant to TSWV, to produce progeny seeds;
b. planting the progeny seeds under conditions favorable for the growth of peanut plants to produce progeny plants;
c. selecting progeny plants that express resistance to TSWV, to produce selected progeny plants;
d. crossing the selected progeny plants with the ‘IPG 913’ plant or the plant of the second peanut line to produce new progeny plants;
e. selecting the new progeny plants that express the TSWV resistance; and
f. repeating steps (d) and (e) three or more times in succession, to produce selected higher filial generation backcross progeny plants that comprise the TSWV resistance.
